(19) United States
(12) Patent Application Publication
(10) Pub. No.: US 2018/0344991 A1
(43) Pub. Date: Dec. 6, 2018

(54) BALLOON CATHETER DEVICE FOR IN VIVO POLYMERIZATION OF BIORESORBABLE SCAFFOLDS

(71) Applicant: Cook Medical Technologies LLC, Bloomington, IN (US)

(72) Inventors: Alec E. Cerchiari, Bloomington, IN (US); Sean D. Chambers, Bloomington, IN (US)

(21) Appl. No.: 15/992,673

(22) Filed: May 30, 2018

Related U.S. Application Data
(60) Provisional application No. 62/513,644, filed on Jun. 1, 2017.

(51) Int. Cl. A61M 25/10; A61B 17/12; A61F 2/958; A61F 2/945

(52) U.S. Cl. CPC A61M 25/1002; A61B 17/12136; A61B 17/12109; A61F 2/945; A61M 2025/105; A61F 2/958

(57) ABSTRACT

Disclosed herein is a device, system, and method for making a bioresorbable intraluminal support device. The intraluminal support device may be introduced to a body lumen of a patient as a prepolymeric substance, and may be polymerized to form the solid device in vivo. The polymerization may be via irradiation, such as ultraviolet irradiation. Methods of treating a patient are also disclosed.

### BACKGROUND

The present application generally relates to medical devices. More particularly, the present application relates to device assemblies for the implantation of bioresorbable scaffolds within a body vessel, such that the body vessel is supported and kept open.

Stents and other implantable medical devices which incorporate stents are in widespread use in the medical field for dilating patient's vessels, for closing off aneurysms, for treating vascular dissections, for supporting prosthetic elements and so on. Stents have the function of holding the vessel open or for holding a device securely against the vessel wall to effect a good seal as well as to prevent device migration. As a result, it is desirable for the stent to be able to apply a significant opening force and to do so reliably. However, in many instances it is undesirable to maintain pressure against the vessel wall for prolonged periods of time. Long term pressure applied to the vessel wall can cause undesired long term effects such as stretching or straightening of the vessel. It is also, in some instances, undesirable to retain long term in a patient's body significant amounts of metal or alloy. These effects can lead to vessel weakening, stenosis or restenosis, and other undesirable conditions.

It is generally undesirable to remove the stent or other medical device from a patient by means of an invasive medical procedure. Therefore, devices that are made of biodegradable or bioresorbable materials are of interest for use in medical devices. While such scaffolds can resolve the problems encountered during the long term use of such implantable medical devices, biodegradable or bioresorbable devices do not generally have the same performance characteristics as metallic stents, resulting in potential loss of efficacy. Furthermore, because many bioresorbable materials lack the elastic properties of metal alloys, delivery of devices that have good patency with vessel walls, and that can readily spring from a collapsed delivery configuration to an expanded configuration is a hurdle that must be overcome.

In general, the development of a novel polymeric bioresorbable device is challenging because candidate polymeric materials must concomitantly include mechanical traits such as high elastic modulus (to impart radial stiffness), large-break strains (to impart the ability to withstand deformations from the crimped state to the expanded state), and low yield strains (to reduce the amount of recoil and overinflation necessary to achieve a target deployment.) Thus, it is not surprising that, in general, in order to provide sufficient hoop strength to oppose negative arterial remodeling and limit acute recoil, polymeric bioresorbable scaffolds have considerably thicker struts (from about 150-200 microns) compared to bare metal or drug-eluting devices (about 80 microns, typically); in order to accommodate the crimping process, polymeric bioresorbable scaffolds have considerably larger crossing profiles (about 1.4 to about 1.8 millimeters (mm)) compared to bare metal and drug eluting devices (about 1.0 mm); and in order to avoid radial force loss and fracture, polymeric bioresorbable scaffolds be appropriately sized (that is, not over-stretched during delivery and deployment.)

It has been a challenge to develop bioresorbable devices for use in a body lumen that have favorable properties and are delivered in a straightforward manner without the need for expanding a preconstructed frame-type structure.

### SUMMARY

In one aspect, the present disclosure provides a medical device including an inflatable element. The inflatable element includes a balloon having an inner surface and an outer surface, and a cover disposed on one of the inner surface and the outer surface. The cover includes a plurality of transmissive segments and a plurality of opaque regions. The transmissive segments may be in interconnected arrangement and bounded by, or surrounded by, or contained within or between the opaque regions.

In another aspect, the present disclosure provides a medical device assembly for delivery of an intraluminal support device. The medical device assembly includes a catheter having a first end and extending to a second end, the catheter defining a central lumen therethrough. The assembly also includes an inflatable element disposed about the catheter. The inflatable element includes a balloon having an inner surface and an outer surface, and a cover disposed on one of the inner surface and the outer surface. The cover includes a plurality of transmissive segments and a plurality of opaque regions. The transmissive segments may be in interconnected arrangement and bounded by the opaque regions.

In another aspect, the present disclosure provides method of making a medical device for forming a biodegradable scaffold. The method includes steps of forming a transmissive portion on opaque layer to define a patterned layer; disposing the patterned layer on an expandable balloon to form an inflatable element; disposing a curable material over the inflatable element; and curing the curable material to form the biodegradable scaffold. The method may optionally include that the inflatable element is disposed circumferentially about and in fluid communication with a catheter. The pattern may be formed on the outer surface of the inflatable element. The inflatable element may include a plurality of pores formed therethrough, and the catheter comprises a second lumen in fluid communication with the pores for delivery of fluid therethrough. The curable material may be curable by radiation; in certain cases, ultraviolet radiation.

Further objects, features and advantages of this system will become readily apparent to persons skilled in the art after a review of the following description, with reference to the drawings and claims that are appended to and form a part of this specification.

### BRIEF DESCRIPTION OF THE DRAWINGS

FIG. 1 is a side view of an inflatable element over a partion of a device assembly constructed in accordance with the principles of the present invention;

FIG. 2 is a view of the device assembly of FIG. 1 illustrated with a layer of a prepolymeric material overlaying the inflatable element;

FIG. 3 is a cross-sectional view of the assembly of FIG. 1 taken across line 3;

FIG. 4 is a cross-sectional view of the assembly of FIG. 2 taken across line 4;

FIG. 5 is a partial cross-section view of an inflatable element and a radiation source demonstrating the transmission and absorption of radiation thereby;

FIG. 6 is a schematic view of a medical device assembly including an inflatable element constructed in accordance with the principles of the present disclosure;

FIG. 7 is a side view of another embodiment of an inflatable element and catheter constructed in accordance with the principles of the present disclosure;

FIG. 8 is a cutaway view of the assembly of FIG. 7 showing interior components;

FIG. 9 is a schematic view of the steps of a method for making a device assembly of the present disclosure; and

FIG. 10 is a schematic view of the steps of a method for using a device assembly of the present disclosure to form a scaffold in the vasculature of a patient in need thereof.

### DETAILED DESCRIPTION

The drawings are purely schematic illustrations of various aspects of the invention and are not necessarily to scale, unless expressly stated.

The terms “substantially” or “about” used herein with reference to a quantity includes variations in the recited quantity that are equivalent to the quantity recited, such as an amount that is equivalent to the quantity recited for an intended purpose or function. “Substantially” or derivatives thereof will be understood to mean significantly or in large part.

One potential solution for bypassing the material constraints associated with crimping and deploying a bioresorbable scaffold without overstretching or fracturing the polymeric scaffold structure can be realized by polymerizing the scaffold in vivo.

In one embodiment, the approach utilizes a balloon catheter that can delivery a photocurable prepolymer composition that polymerizes at the desired site of deployment upon exposure to electromagnetic radiation, such as ultraviolet radiation. In some embodiments, this may be achieved by the use of an inflatable element which includes a patterned balloon that can selectively block or transmit electromagnetic radiation through its surface, and a photocurable prepolymeric composition that polymerizes upon exposure to this transmitted radiation.

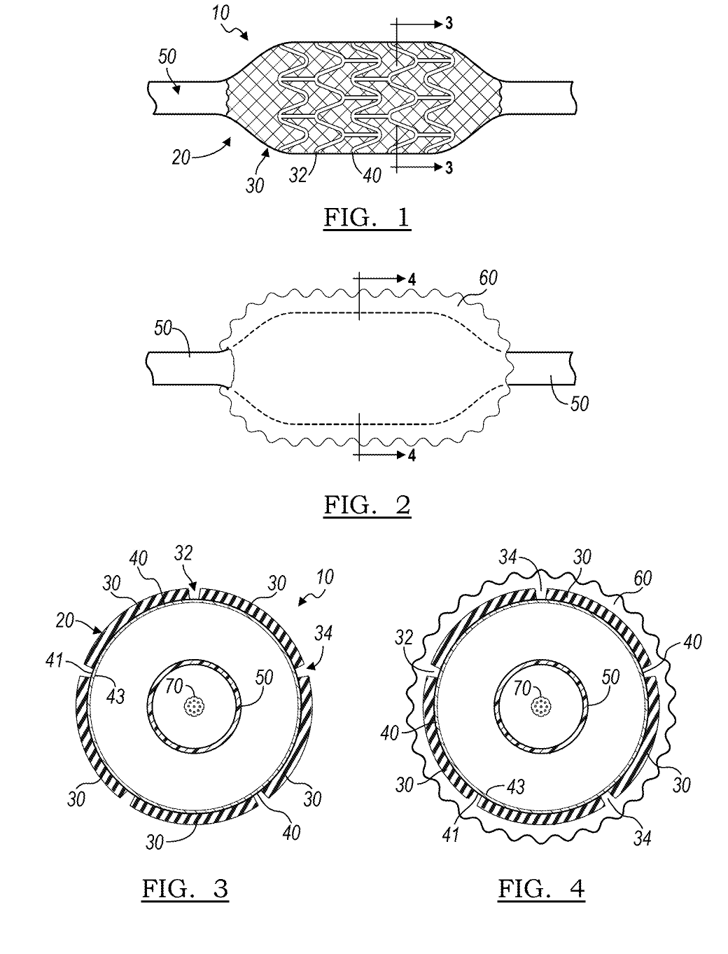

FIG. 1 illustrates a balloon catheter 10 that includes an inflatable element 20. The inflatable element 20 surrounds the catheter body 50 circumferentially, and is made of two layers: an inner balloon 40, with a cover, or shield, 30 surrounding the inner balloon 40. Inner balloon 40 is visible through the spaces 34 in pattern 32 formed in shield (or cover) 30. The inner balloon 40 is not visible through shielded portion 36 of the shield 30.

The pattern 34 is, in the illustrated embodiment, an interconnected pattern of struts and bends resembling a stent or scaffold. The struts and bends are interconnected so as to form a single structure which, after polymerization and solidification, may give rise to a single, unitary medical implant. As shown in the drawings, the transmissive segments are shown to be in interconnected arrangement and bounded by the opaque regions. The pattern allows for a scaffold to be cured and solidified within a body lumen. It is within the scope of this disclosure that a pattern might be formed in a shield 30 so as to result in the formation of a plurality of medical implants instead, if desired.

The inner balloon 40 is, in one embodiment, a standard inflatable balloon for a balloon catheter. The inner balloon 40 should uniformly diffuse electromagnetic radiation generated from inside of the balloon through its surface. The radiation from the probe within the balloon may be delivered to the entire circumferential profile of the inner balloon 40. In one embodiment, the inner balloon 40 may be made of an optically transparent polyurethane. Other materials are also suitable, such as a nylon, a rubber, an impervious polymer, and so forth.

The radiation source, or illuminator, of the device assembly may be modified or enhanced to scatter light (or other radiation) such that the majority, or nearly all, of the provided radiation is directed to the circumference and the balloon wall rather than allowing the light (or other radiation) to continue along a path along the longitudinal axis and out of the distal end of the assembly. Alternatively or additionally, the shield 30 may be patterned such that the distal end of the inner balloon 40 is covered, so that radiation is not transmitted substantially along the axis of the lumen.

In one embodiment, the shield or cover 30 may be a balloon with radiopaque characteristics. In other embodiments, the cover 30 may be a plurality of separate opaque sections laid on at least one of an inner surface and an outer surface of the balloon.

FIG. 2 illustrates the inflatable element 20 of FIG. 1 covered with a prepolymeric material 60. The prepolymeric material 60 is a substance which, when exposed to a polymerizing agent, will rapidly solidify and cure to a final device shape at the site of implantation. The polymerizing agent may, in some embodiments, by a chemical catalyst, including components of the luminal fluid that the prepolymeric material encounters at deployment. In another embodiment, the polymerizing agent is electromagnetic radiation from a radiation probe housed within the inner cavity of the inner balloon 40. The radiation may take the form of ultraviolet light, infrared light, blue light, yellow light, or any other form of transmittable radiation. When ultraviolet irradiation is employed, the intensity of the radiation may be above 100 millijoules per square centimeter (mJ/cm²), or between 100 mJ/cm² and 250 mJ/cm², or about 150 mJ/cm², or about 200 mJ/cm², or between 150 mJ/cm² and 200 mJ/cm². The radiation probe/diffuser may be formed in any suitable shape, including a cylinder, a conical element, or the like.

The prepolymeric material (or polymeric precursor) is a biocompatible and biodegradable (or bioresorbable) material. Examples of materials that may be used include a poly(lactic acid), a poly(glycerol-co-sebacate), a poly(ethylene glycol), a poly(propylene fumarate), a polycaprolactone, a poly(alpha-hydroxy ester), a poly(beta-amino ester), a polyvinyl, and a polysaccharide, among others.

In some embodiments, the prepolymeric material 60 is a viscous material. The viscosity allows the material to adhere to the outer surface of the inflatable element 20 and to remain on the outer surface during deliver of the material to the site of treatment. In some embodiments, the inflatable element is delivered to the site of treatment in such a way that the prepolymeric material 60 represent the outermost layer of the assembly, contacting the wall of the body lumen directly when the inflatable element 20 is inflated to its expanded state. In some embodiments, the viscosity of the prepolymeric material will be at least 100 pascal-seconds, such that it principally remains adhered to the delivery assembly during delivery.

FIG. 3 and FIG. 4 are cross sectional views of the device assembly 10 as illustrated in FIGS. 1 and 2, respectively. FIG. 3 shows the radiation probe 70 within the interior of inner balloon 40. The radiation probe 70 is fed into the balloon 40 via catheter 50. The cross-sectional view of FIGS. 3 and 4 is taken across a portion of the device assembly 10 in which the pattern 32 of the shield 30 is present. As can be seen in FIG. 3, the pattern 32 leaves gaps 34 in shield 30, making portions of the outer surface of inner balloon 40 the effective outermost layer of the device prior to application of the prepolymeric composition.

As illustrated in FIG. 4, the device assembly 10, when coated with prepolymeric material 60, is in large part an assembly in which the prepolymeric material 60 is in contact with the shield 30. However, portions of the inner balloon 60 are also in direct contact with the prepolymeric composition. It is the prepolymeric material that is exposed to radiation that is transmitted through the spaces 34 of pattern 32 that will cure and become solidified when the device is formed in vivo.

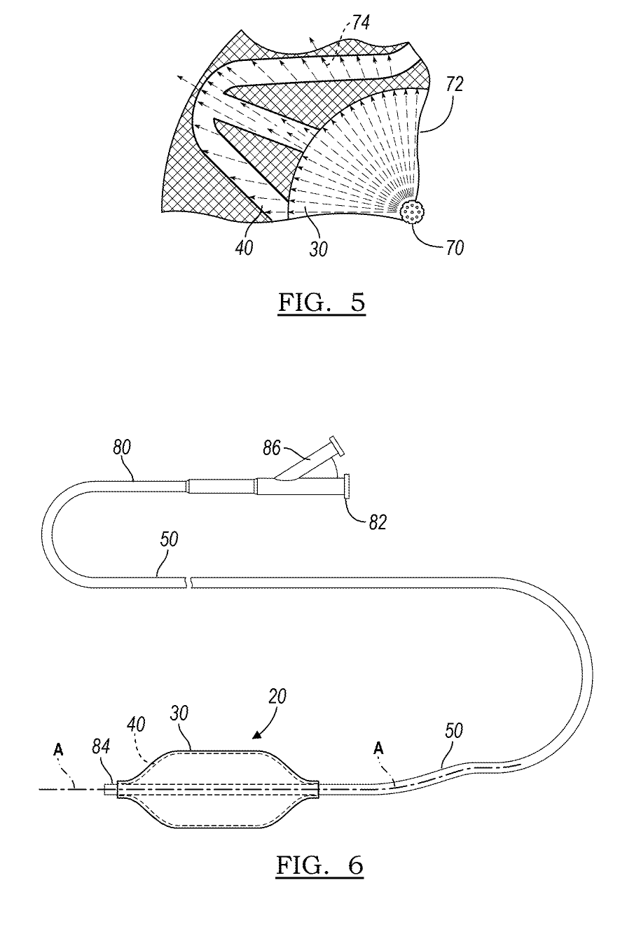

FIG. 5 is a schematic illustration of the transmission of radiation from the radiation source 70 through the gaps 34 of the pattern 32 formed in shield 30. Because the shield 30 is made of radiopaque sections, some of the radiation 72 is blocked from contacting the prepolymeric material. However, the gaps in pattern 32 allow for a certain quantity and pattern of transmitted radiation 74 to escape through inner balloon 40, thereby allowing the prepolymeric material to be exposed, in a defined pattern, such that the structure of the solid implant is formed as the exposed portions of the prepolymeric layer are cured.

FIG. 6 is an illustration of a catheter assembly 80 that incorporates the shielded inflatable element 20 of the present disclosure. The assembly 80 extends from a proximal end 82, which may be attached to a handle, and comprise ports 86 and 88 for the delivery of fluid (including the prepolymeric fluid, and inflation fluid for the inflatable element), to distal end 84, which defines a distal portion of the assembly 80 at which the inflatable element 20 is positioned. The catheter body 50 defines longitudinal axis A along its length.

In one embodiment, the prepolymeric material may be deposited on the outer surface of the inflatable element of the device. In another embodiment, a weeping balloon catheter 110, as illustrated in FIG. 7, may be used instead, delivering the prepolymer through the pores 190 in the outer surface of the inflatable element 120. The shield 130 includes a pattern 132 for the scaffold to be formed, as well as radiopaque shielded portions 136 through which radiation is not transmitted.

Weeping balloon catheters are generally used for drug delivery and generally include a catheter having an elongate tubular body defining an inflation lumen and a combination lumen that terminates at an open distal end of the elongate tubular body. A dilation balloon is disposed at a distal end of the elongate tubular body and is in fluid communication with the inflation lumen. A drug delivery balloon is disposed at least partially over the dilation balloon and includes at least one drug release opening through an outer wall of the drug delivery balloon. The drug delivery balloon is in fluid communication with the combination lumen through a sidewall opening of the elongate tubular body. Such balloons are described in U.S. Pat. Nos. 8,034,022, 8,591,461, 8,911,399, 9,028,443, and 9,174,030, all of which are incorporated herein by reference in their entireties.

FIG. 8 is a cutaway view of a weeping balloon catheter assembly 110 incorporating an inflatable element 120 usable in accordance with principles of the present disclosure. The outer catheter 150 is attached to and surrounded by the inflatable element 120.

The outer catheter 150 contains four lumens. Inner catheter 152 is attached to the inflatable element 120, both shield 130 and inner balloon 140. Inner catheter 152 may be a solid support element to impart stiffness to the device, or may house a wire guide lumen so that the assembly may be delivered over a wire guide.

Injection lumen 154 opens into, and is in fluid communication with, the interior of inner balloon 140, and is used to deliver and withdraw inflation fluid to convert the inflatable element 120 between its expanded (or inflated) and contracted (or deflated) states. Source lumen 156 houses radiation probe 170. Source lumen 156 is an optional structure; in another embodiment, if the inner catheter 152 is made of a transmissive material, the probe 170 may be delivered in, and housed within, its lumen.

Prepolymer lumen 158 terminates within the shield 130 delivers precursor polymer through the pores 190 in shield to coat the exterior surface of inflatable element 120. The prepolymer lumen 158 terminates outside of the inner balloon 140.

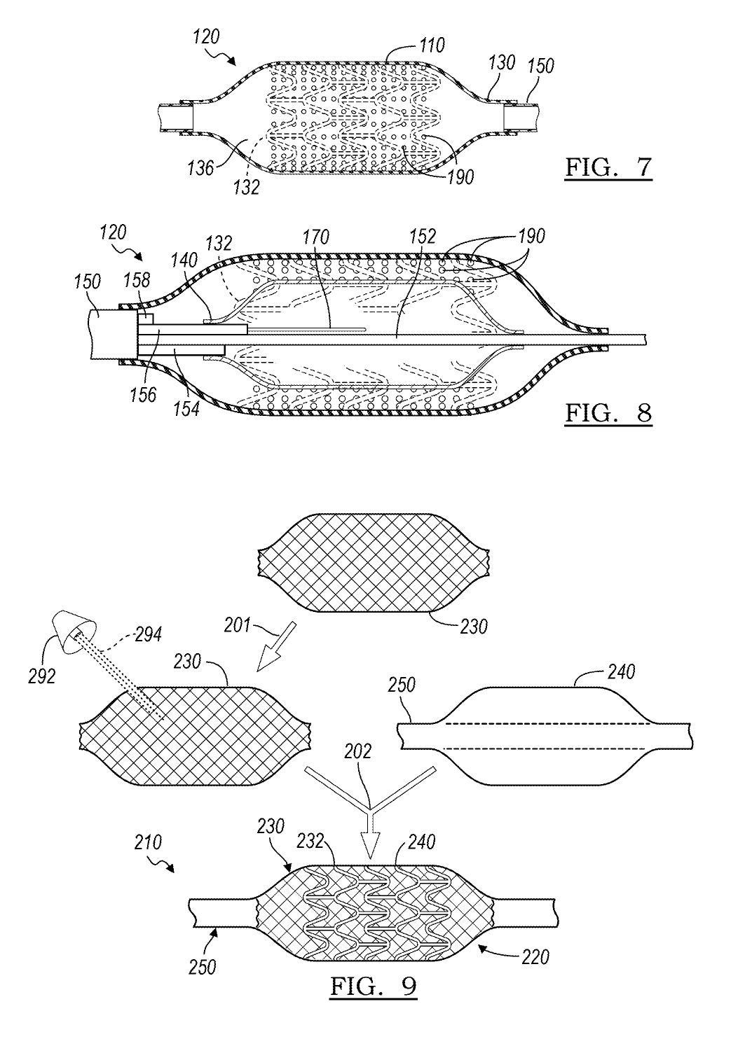

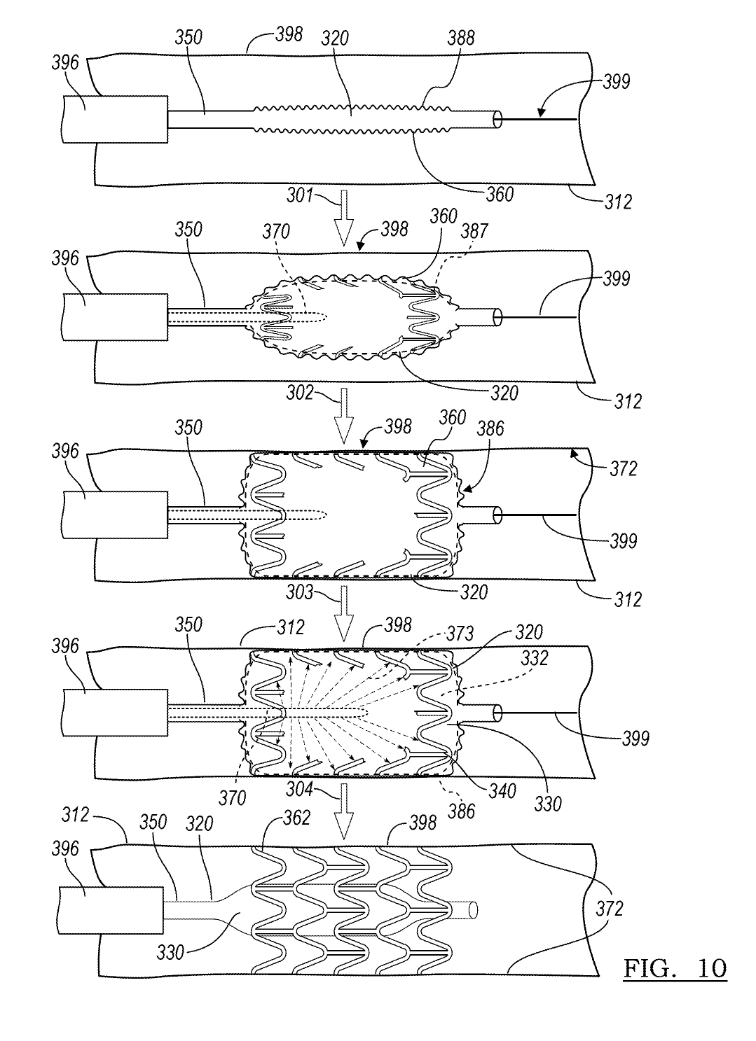

FIG. 9 illustrates steps of making a balloon catheter assembly 210 with an inflatable element 220 constructed in accordance with the principles of the present disclosure. A standard balloon catheter with elongate tubular catheter body 250 may be used, so long as the balloon 240 is transmissive to the type of radiation intended to be used to cure the prepolymeric material into the final solid implant. The balloon 240 is disposed circumferentially about the catheter body 250, and is in fluid communication with at least a portion of the lumen which runs through and is defined by catheter body 250, so as to permit for the balloon to be inflated by inflation fluid.

In step 201, a shield 230 is provided, and processed in order to generate a pattern 232 therein. The processing step may including using a laser 292 to emit a beam 294 which is capable of cutting the material of shield 230, allowing for the pattern to be cut away from the remainder of the material. The shield 230 may be a balloon which has been constructed to have radiopaque qualities. For example, the polymer from which the shield 230 is constructed may be doped with a radiopaque material, such as bismuth, tungsten, another metal, or a radiopaque polymer, and then formed into a balloon shape. The shield is provided as a generally uniform, monolithic, balloon shaped cylinidrical element.

In a second step 202, the inflatable element 220 is constructed. The inflatable element 220 includes shield 230, having pattern 232, disposed about inner balloon 240. At least the inner portion of inflatable element 220, including inner balloon 240, is in fluid communication with the lumen of catheter 250 to form a balloon catheter.

Although the method of FIG. 9 demonstrates creating the radiopaque layer external to the inner balloon 240, or about the outer surface of inner balloon 240, it will be appreciated that the radiopaque patterned layer may be formed in a number of different ways. For example, a patterned balloon may instead be placed within the interior of an effectively fully-transmissive outer balloon. In another embodiment, it is possible to use a single balloon which is manufactured to have a generally transmissive body but for the presence of a radiopaque pattern thereon or therein. Such a balloon may be created by, for example, an additive manufacturing, or a coextrusion process. It will be understood that in an assembly in which the inflatable element consists of a single inflatable balloon, that the prepolymeric fluid will be disposed over the external surface of the single balloon, rather than over an shield component.

In some embodiments, the assembly may be prepackaged with the prepolymeric material disposed over the inflatable element. In other embodiments, the operator of the device may dispose the prepolymeric material over the inflatable element prior to conducting the delivery procedure.

In one embodiment, the cover or shield may be patterned prior to overlaying the inner or outer surface of the inner balloon. In another embodiment, the intact cover or shield may be overlaid over the inner balloon and then patterned, such as by laser cutting.

FIG. 10 illustrates a method of using an assembly in accordance with the principles of the present disclosure to deliver and implant a medical device within a body lumen. In the first step, catheter 350 is moved through delivery sheath 396 and the inflatable element 320, coated with prepolymer 360 and in its deflated state, is advanced to the treatment site 398 of body vessel 312. Optionally, the assembly is advanced over wire guide 399. Suitable body vessels include blood vessels, such as veins and arteries, as well as lumens of the digestive system (such as the esophagus), the respiratory system, and any portion of the anatomy that may need to be kept open and supported by a scaffold. One particular use for this device and method includes stenting occlusions within the superficial femoral artery.

In step 301, inflation fluid is provided to the inflatable element 320 such that it takes on a partially inflated state 387.

In step 302, the inflation of inflatable element 320 is complete, leading to completely inflated state 386. At this point, the prepolymeric material 360 has contacted the inner wall 372 of body vessel 312.

In step 303, the radiation source is activated and the probe 370 distributes electromagnetic radiation through the inner balloon 340, and through the pattern 332 of shield 330, to selectively solidify and cure portions of the prepolymeric coating of the inflatable element 320. Such an irradiating step lasts as long as necessary to achieve the solidification of the portions of the prepolymer 360. The entirety of pattern 332 is not shown in the drawings for the sake of simplicity and clarity.

In step 304, the device 362 has been formed from the viscous liquid precursor. A solid implant has been formed in vivo. It is sized appropriately to the vessel because the prepolymer 360 had been in contact with the vessel wall 372. The inflatable element 320 is deflated by withdrawing the inflation fluid through the inflation lumen, thus collapsing the balloon 340 and the shield 330. Some prepolymer may remain on the inflatable element 320 and may likewise be withdrawn from the patient. However, some unpolymerized material may also remain in the body lumen. Because the polymers to be used in such a method are bioresorbabale, the unpolymerized substance will simply dissolve within the vessel.

As a person skilled in the art will readily appreciate, the above description is meant as an illustration of implementation of the principles this application. This description is not intended to limit the scope of this application in that the system is susceptible to modification, variation and change, without departing from the spirit of this application, as defined in the following claims.

What is claimed is:

1. A medical device comprising:
an inflatable element comprising:
a balloon having an inner surface and an outer surface, and
a cover disposed on one of the inner surface and the outer surface, the cover comprising a plurality of opaque regions and defining a plurality of transmissive segments, the transmissive segments being in interconnected arrangement.

2. The inflatable element of claim 1, wherein the cover is disposed over the outer surface of the inflatable element.

3. The inflatable element of claim 1, wherein the cover is disposed over the inner surface of the inflatable element.

4. The inflatable element of claim 1, wherein the medical device is a balloon catheter.

5. A medical device assembly for delivery of an intraluminal support device, the medical device assembly comprising:
a catheter having a first end and extending to a second end, the catheter defining a central lumen therethrough; and
an inflatable element disposed circumferentially about the catheter and in fluid communication with the central lumen, the inflatable element comprising:
a balloon having an inner surface and an outer surface, and
a cover disposed on one of the inner surface and the outer surface, the cover comprising a plurality of opaque regions and defining a plurality of transmissive segments, the transmissive segments being in interconnected arrangement.

6. The inflatable element of claim 5, wherein the cover is disposed over the outer surface of the inflatable element.

7. The inflatable element of claim 5, wherein the cover is disposed over the inner surface of the inflatable element.

8. The medical device assembly of claim 5, further comprising a coating of a prepolymeric material disposed over the outer surface of the inflatable element.

9. The medical device assembly of claim 5, wherein the prepolymeric material is curable by irradiation.

10. The medical device assembly of claim 9, wherein the prepolymeric material at least one selected from the group consisting of a poly(lactic acid), a poly(glycerol-co-sebacate), a poly(ethylene glycol), a poly(propylene fumarate), a poly(alpha-hydroxy ester), a poly(beta-amino ester), a polyvinyl, and a polysaccharide.

11. The medical device assembly of claim 5, wherein the inflatable element comprises a plurality of pores formed therethrough, and the catheter comprises a second lumen in fluid communication with the pores for delivery of fluid therethrough.

12. The medical device assembly of claim 8, wherein when the assembly is housed within a delivery sheath, the prepolymeric material is an outermost element of the assembly.

13. The medical device assembly of claim 5, further comprising a radiation source.

14. The medical device assembly of claim 13, wherein the radiation source produces ultraviolet radiation.

15. A method of making a medical device for forming a biodegradable scaffold, the method comprising:
forming a transmissive portion of an opaque layer to define a patterned layer; and
disposing the patterned layer on an expandable balloon to form an inflatable element.

16. The method of claim 15, further comprising disposing a curable material over the inflatable element.

17. The method of claim 15, wherein the inflatable element is disposed circumferentially about and in fluid communication with a catheter.

18. The method of claim 15, wherein the pattern is formed on the outer surface of the inflatable element.

19. The method of claim 15, wherein the inflatable element comprises a plurality of pores formed therethrough, and the catheter comprises a second lumen in fluid communication with the pores for delivery of fluid therethrough.

20. The method of claim 16, wherein the curable material is curable by radiation.

21. The method of claim 16, further comprising curing the curable material to form the biodegradable scaffold.
